(19) United States
(12) Patent Application Publication
(10) Pub. No.: US 2016/0183886 A1
(43) Pub. Date: Jun. 30, 2016

(54) BIOLOGICAL INFORMATION PREDICTING APPARATUS AND BIOLOGICAL INFORMATION PREDICTING METHOD

(71) Applicant: NIHON KOHDEN CORPORATION, Tokyo (JP)

(72) Inventor: Norihito KONNO, Tokyo (JP)

(21) Appl. No.: 14/973,280

(22) Filed: Dec. 17, 2015

(30) Foreign Application Priority Data
Dec. 24, 2014 (JP) 2014-260695

(51) Int. Cl. A61B 5/00; A61B 5/024

(52) U.S. Cl. CPC A61B 5/7275; A61B 5/024; A61B 5/746

(57) ABSTRACT

A biological information predicting apparatus and a biological information predicting are provided. The biological information predicting apparatus includes a biological parameter acquiring section configured to acquire a first biological parameter and a second biological parameter, a biological information predicting section configured to predict a future trend of the second biological parameter based on a future prediction model and a history of values of the first biological parameter acquired by the biological parameter acquiring section, the future prediction model defining a relationship between a change of the first biological parameter and a change of the second biological parameter, and a notifying section configured to provide a notification related to the second biological parameter based on the prediction by the biological information predicting section.

### CROSS-REFERENCE TO RELATED APPLICATION(S)

The present application claims priority from Japanese Patent Application No. 2014-260695 filed on Dec. 24, 2014, the entire content of which is incorporated herein by reference.

### BACKGROUND

The presently disclosed subject matter relates to a biological information predicting apparatus and a biological information predicting method.

Recently, an aging society is becoming a serious problem worldwide. Particularly in Japan, the problem of aging is quite significant. It is said that the social security in Japan will be shifted from a structure in which one aged person is supported by three or four people of productive age, to a structure in which one aged person is supported by one person of productive age. In such a social structure, it is necessary to consider particularly the following points.

Firstly, when the percentage of aged people is increased, there is a possibility that medical expenses are remarkably increased. Therefore, it is important to rapidly treat a patient of disease and soon discharge (restore) the patient from hospital. Secondly, from the viewpoint of utilization of aged people, it is important not to put an aged person in hospital (not to cause an aged person to become sick). As a countermeasure against the two points, it is critical to immediately assess the risk of a disease before the disease becomes worse.

The recent improvement in processing power of a computer enables a large volume of data in a wide variety of formats to be handled at high velocities. In such a circumstance, various analyzing methods and techniques such as machine learning and data mining are used in various fields. Also in the medical field, studies have been made to use these techniques in disease prediction and the like.

The related art for predicting the risk of a disease by using statistical analysis or the like will be described. According to a first related art, an apparatus is configured to compare saliva data acquired from the subject with previously stored correlation data, to determine a lifestyle disease (see, e.g., JP2014-130096A). According to a second related art, a correlation between the body weight of a subject and medical examination data (total cholesterol and the like) is analyzed, and the health condition is estimated from the result of the analysis (see, e.g., JP2009-181564A).

According to the first related art, the risk of a lifestyle disease at the time of the acquisition of the saliva data is determined by comparing the saliva data with the correlation data. According to the second related art, the health condition at the time of medical examination is determined based on the body weight. That is, in both cases, the risk of a disease or the health condition at a certain timing is analyzed based on relationships (correlation) of a plurality of biological parameters. In other words, a future risk of a disease or the like cannot be predicted in advance. Recently, efforts to predict a future of a patient are gradually being made. However, details of processes of such prediction are not disclosed, and sufficient studies have not been made.

Examples of diseases in which advanced prediction is desired are cardiac arrest, at-risk arrhythmia such as ventricular fibrillation, heart rate change, etc. In related art biological information monitors, an electrocardiogram is monitored and analyzed to detect such an at-risk condition, and the detection is informed by providing an alarm or the like. However, an alarm provided by general biological information monitors does not inform of future risk. Therefore, it is desired to obtain biological information at an earlier stage.

### SUMMARY

Illustrative aspects of the present invention provide a biological information predicting apparatus and a biological information predicting method, which can predict future biological information.

According to an illustrative aspect of the present invention, a biological information predicting apparatus includes a biological parameter acquiring section configured to acquire a first biological parameter and a second biological parameter, a biological information predicting section configured to predict a future trend of the second biological parameter based on a future prediction model and a history of values of the first biological parameter acquired by the biological parameter acquiring section, the future prediction model defining a relationship between a change of the first biological parameter and a change of the second biological parameter, and a notifying section configured to provide a notification related to the second biological parameter based on the prediction by the biological information predicting section.

### BRIEF DESCRIPTION OF DRAWINGS

FIG. 1 is a block diagram of a configuration of a biological information predicting apparatus according to one or more exemplary embodiments of the present invention;

FIG. 2 is a block diagram of a configuration of a biological information predicting apparatus according to an exemplary embodiment of the present invention;

FIG. 3 is a diagram showing a relationship between pulse rates (PR) and heart rates (HR) acquired by a biological parameter acquiring section of the biological information predicting apparatus;

FIG. 4 is a chart showing a concept of operations of an error excluding section of the biological information predicting apparatus of FIG. 2;

FIG. 5 is an example of a box plot chart used by an outlier excluding section of the biological information predicting apparatus of FIG. 2;

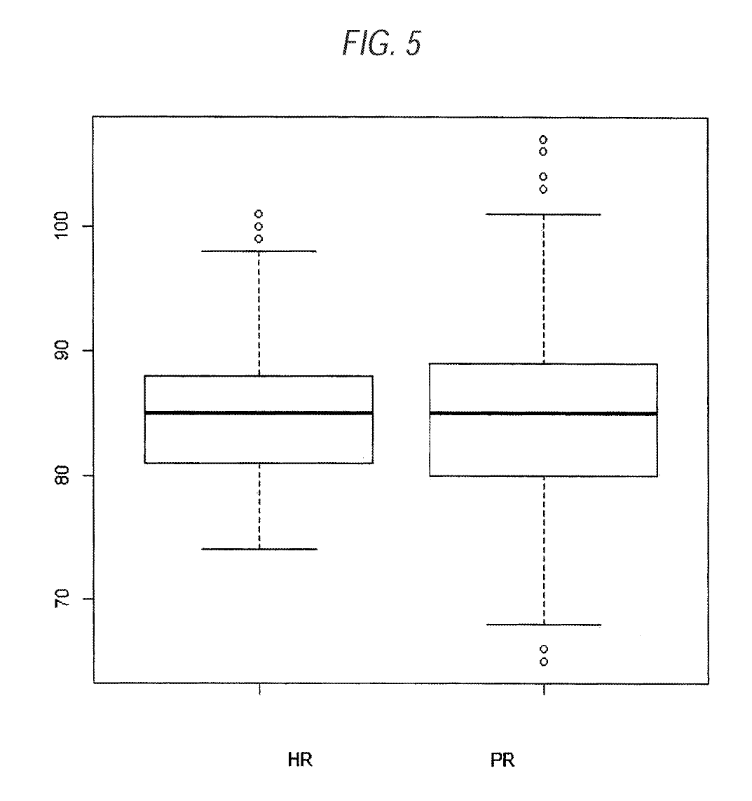

FIG. 6 is a block diagram of a configuration of a biological information predicting apparatus according to another exemplary embodiment of the present invention; and

FIG. 7 is a chart showing an example of a future prediction model stored in a storage section of the biological information predicting apparatus of FIG. 6.

### DETAILED DESCRIPTION

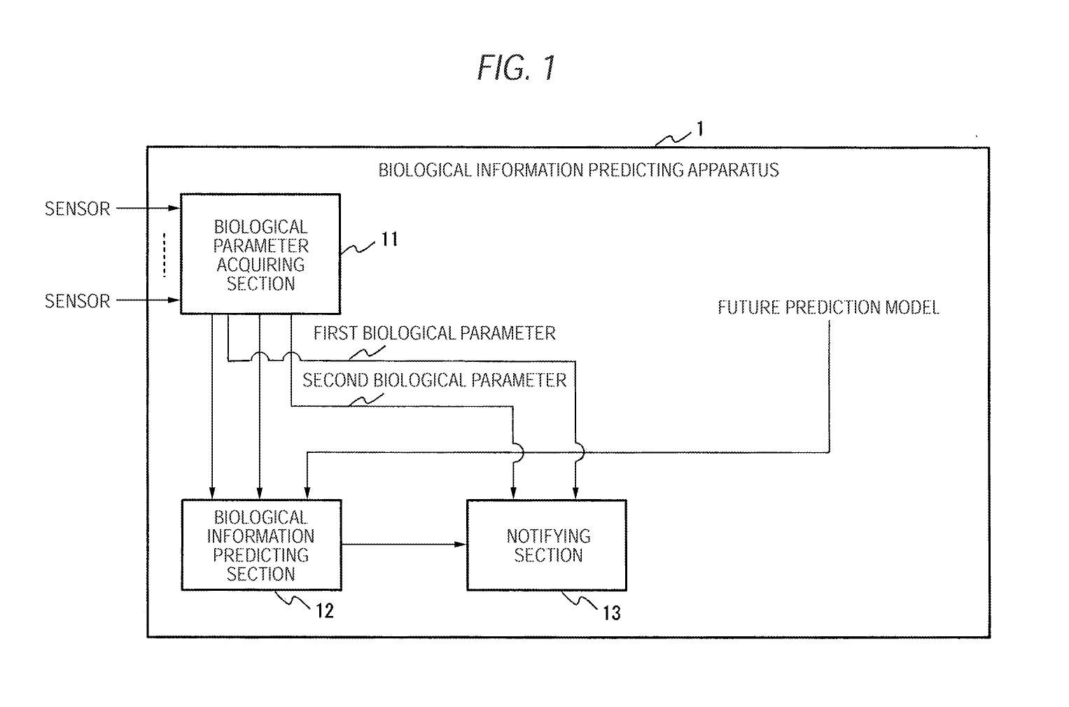

FIG. 1 is a block diagram of a configuration of a biological information predicting apparatus 1 according to one or more exemplary embodiments of the present invention. The biological information predicting apparatus 1 includes a biological parameter acquiring section 11, a biological information predicting section 12, and a notifying section 13. The biological parameter acquiring section 11 measures the blood pressure, the respiratory rate, the respiratory waveform, the body temperature, the pulse rate, the pulse waveform, the heart rate, an electrocardiogram, the artery oxygen saturation, and the like. For example, the biological information predicting apparatus 1 is a bedside monitor, a defibrillator, a transmitter, or the like. Although not illustrated, the biological information predicting apparatus 1 further includes a central processing unit (CPU), various storage devices (a primary storage device and a secondary storage device), a displaying device (a liquid crystal display), and the like.

The biological parameter acquiring section 11 acquires measurement values of biological parameters from various sensors (electrodes, probes, a cuff, and the like) attached to the subject. The biological parameter acquiring section 11 acquires at least one or more biological parameters (a first biological parameter). Preferably, the biological parameter acquiring section 11 acquires two or more biological parameters (a first biological parameter, a second biological parameter, a third biological parameter, etc.). In the following description, it is assumed that the first biological parameter is the pulse rate (PR), and the second biological parameter is the heart rate (HR). The biological parameter acquiring section 11 supplies measurement values of the acquired biological parameters to the biological information predicting section 12, and the notifying section 13.

The biological information predicting section 12 predicts a future trend of the heart rate (HR) based on a history (transition) of the measured values of the pulse rate (PR) acquired by the biological parameter acquiring section 11, and a future prediction model. For example, the future trend of the heart rate (HR) (second biological parameter) may be a specific numeric value such as “value of the heart rate (HR) at five minutes after” or information indicating an approximate condition such as “the heart rate (HR) may have an abnormal value at five minutes after.”

Preferably, the biological information predicting section 12 may predict a future risk level of the heart rate (HR) based on the history (transition) of the measured values of the pulse rate (PR) acquired by the biological parameter acquiring section 11, and the future prediction model. The risk level is a level (degree) of risk indicated by, for example, a numerical value. For example, the risk levels may include Level 0 (normal), Level 1 (slightly at-risk), Level 2 (at risk), or Level 3 (highly at-risk). The risk levels need not be discrete data, and may be continuous data.

The future prediction model defines a relationship between a change of the value of the pulse rate (PR) (first biological parameter) and a change of the value of the heart rate (HR) (second biological parameter). For example, the future prediction model may be a regression formula which will be described later in detail in connection with a specific exemplary embodiment. Examples of the regression formula include the following expression (1). The method of calculating the regression formula will be described later.

[symbol]

= [symbol] + [symbol] ×PR (1)

Briefly describing, in the case where the pulse rate (PR) has the following values, the biological information predicting section 12 substitutes 85 as the pulse rate at five minutes after in the expression (1). This causes the biological information predicting section 12 to predict the heart rate (HR) at five minutes after. That is, the biological information predicting section 12 predicts the future of the heart rate (HR) by using the history of values (70, 75, and 80 below) of the pulse rate (PR), and the future prediction model.

Pulse rate (PR) at ten minutes before—70

Pulse rate (PR) at five minutes before—75

Current pulse rate (PR)—80

The biological information predicting section 12 calculates the future risk level from the future value of the heart rate (HR). The biological information predicting section 12 calculates the risk level by comparing a threshold with the future value in such a manner that, when the future value of the heart rate (HR) is smaller than 110, for example, the risk level is calculated as Level 0; when equal to or larger than 110, the risk level is calculated as Level 1; when equal to or larger than 130, the risk level is calculated as Level 2; and, when equal to or larger than 160, the risk level is calculated as Level 3. The risk level is notified by the notifying section 13, and therefore it is possible to know the risk level of the heart rate (HR) of the subject.

The biological information predicting section 12 may set thresholds for setting the risk level, from a history of past values of the heart rate (HR). In the case where the average value during a period when the heart rate (HR) is stabilized (a period when the heart rate is less varied) is 80, for example, the biological information predicting section 12 sets the thresholds to 110, 130, and 160, respectively. In the case where the average value during a period when the heart rate (HR) is stabilized (a period when the heart rate is less varied) is 70, for example, the biological information predicting section 12 sets the thresholds to 100, 120, and 150, respectively.

In the above description, it is assumed that the future prediction model is indicated by an expression. Alternatively, the following definition may be employed as the future prediction model.

The pulse rate (PR) is continuously in an upward trend for five minutes or longer→there is a possibility that the heart rate (HR) becomes abnormal in future (Level 1).

The pulse rate (PR) is increased by 20 or more as compared with the rate at five minutes before→there is a possibility that the heart rate (HR) becomes abnormal in future (Level 2).

The biological information predicting section 12 calculates a future trend (preferably, a risk level) of the heart rate (HR) by using the above-described future prediction model, and supplies the calculated future trend (preferably, the risk level) to the notifying section 13. Alternatively, the biological information predicting section 12 may be configured so as to notify of the future value as it is of the heart rate (HR) in place of the risk level.

Measurement values of various biological parameters acquired by the biological parameter acquiring section 11 are sequentially supplied to the notifying section 13. In the case where the measurement value of a certain biological parameter is an abnormal value, the notifying section 13 outputs an alarm indicating of an abnormality.

Also information of the future prediction (future trend of the second biological parameter) of the heart rate (HR) which is performed by the biological information predicting section 12 is supplied to the notifying section 13. In the case where the future trend of the heart rate (HR) is not normal (where an abnormal value or an abnormal state is expected in the future), the notifying section 13 outputs an alarm even if the current value of the heart rate (HR) is within the normal range. That is, before the second biological parameter shows an abnormal value, the notifying section 13 provides a notification (e.g., an alarm output and a display of a message on the displaying device) related to the second biological parameter based on the future trend of the second biological parameter. Since the notification is provided before an abnormal value appears, a doctor or the like can apply treatment before the condition of the subject gets worse. The notifying section 13 may be configured to directly display the future value of the heart rate (HR).

The notifying section 13 may differentiate the sound of the alarm output in the case where the heart rate (HR) is currently abnormal, from the sound of the alarm output in the case where, although the heart rate (HR) is currently normal, the future trend is abnormal. For example, the former and latter sounds may have different tones. The notifying section 13 may differentiate the blinking color and pattern of a display lamp disposed on the housing in the case where the heart rate (HR) is currently abnormal, from those of the display lamp disposed on the housing in the case where, although the current value is normal, the future trend is abnormal. The alarm sounds may be output at different volumes. That is, the output manner of the notifying section 13 is not limited to only the differentiation in the output of sounds, and the notifying section 13 may differentiate the notifying method in the case where the heart rate (HR) is abnormal, from that in the case where, although the current value is normal, the future trend is abnormal, using different notifying means. When different notifying methods are employed as described above, a doctor or the like can easily determine a countermeasure (e.g., whether a treatment is to be immediately conducted or follow-up observation is to be carefully conducted).

The notifying section 13 may change the notifying method in accordance with the risk level of the future value of the heart rate (HR). The notifying section 13 may change the tone of the alarm sound or the like in accordance with the risk level, or may change the blinking color and pattern of the display lamp disposed on the housing in accordance with the risk level. According to the configuration, a doctor or the like can intuitively notice a degree of a future risk of the subject.

The configuration and operation of the biological information predicting apparatus 1 have been briefly described. Here, advantageous effects of the biological information predicting apparatus 1 described above are will be described. As described above, the biological information predicting section 12 predicts the value of the heart rate (HR) by using the future prediction model that defines the relationship between a change of the pulse rate (PR), an example of the first biological parameter, and a change of the heart rate (HR), an example of the second biological parameter. The notifying section 13 is configured so as to, in accordance with the prediction, perform notification (preferably, with an output of alarm sound) even before the heart rate (HR) becomes abnormal. A doctor or the like refers the notification, and can perform future prediction of biological information that cannot be known with general biological information monitors.

Now, a first specific example of the configuration of FIG. 1 according to an exemplary embodiment of the present invention will be described. By using the first and second biological parameters acquired from the subject, the biological information predicting apparatus 1 produces a future prediction model of the second biological parameter, and predicts the future value of the second biological parameter by using the produced future prediction model. Preferably, the biological information predicting apparatus 1 produces the future prediction model by using regression analysis. Also in the following description, it is assumed that the first biological parameter is the pulse rate (PR), and the second biological parameter is the heart rate (HR). Furthermore, it is assumed that the ST value is used as the third biological parameter.

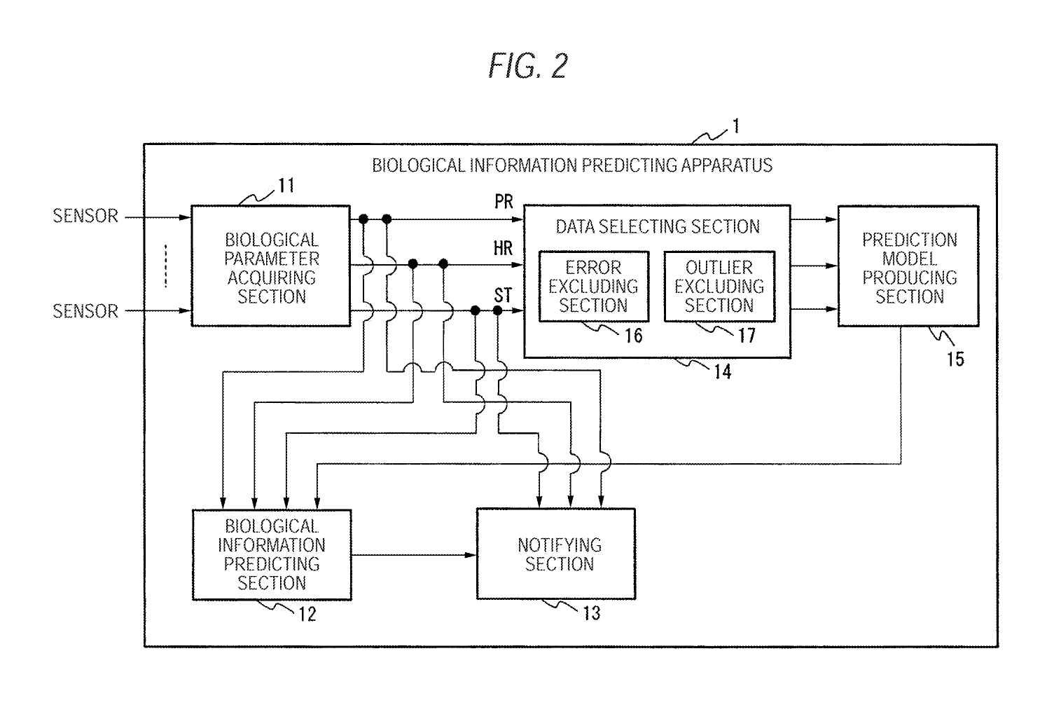

FIG. 2 is a block diagram of a configuration of the biological information predicting apparatus 1 according to the exemplary embodiment. In the following description, the sections designated by the same names and reference numerals as those described above perform the same processes as those described above, unless otherwise described below.

The biological information predicting apparatus 1 includes the biological parameter acquiring section 11, the biological information predicting section 12, the notifying section 13, a data selecting section 14, and a prediction model producing section 15. The data selecting section 14 includes an error excluding section 16 and an outlier excluding section 17.

The biological parameter acquiring section 11 supplies various acquired biological parameters to the data selecting section 14, the biological information predicting section 12, and the notifying section 13. In the present example, the biological parameter acquiring section 11 continuously acquires and supplies the pulse rate (PR), the heart rate (HR), and the ST value. The ST value is the difference between the S wave and the T wave in an electrocardiogram waveform.

Before the model production by the prediction model producing section 15, the data selecting section 14 selects only necessary measurement values from the measurement values of the various biological parameters which are acquired by the biological parameter acquiring section 11, and supplies the selected measurement values to the prediction model producing section 15. This process is performed in order to enhance the accuracy of analysis (preferably, regression analysis) by the prediction model producing section 15.

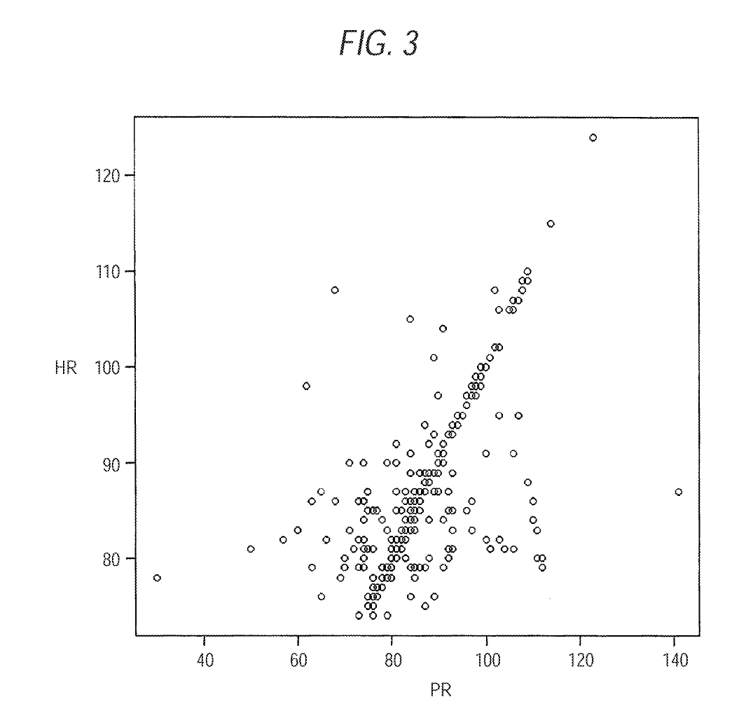

FIG. 3 is a view relationship between the pulse rate (PR) and heart rate (HR) acquired by the biological parameter acquiring section 11. FIG. 3 shows plots of data of every minute in the case where the pulse rate (PR) is plotted on the abscissa, and the heart rate (HR) is plotted on the ordinate. The pulse rate (PR) and the heart rate (HR) are values originating from the motion of the heart of the subject, and therefore preferably have the same value. However, a discrepancy between the values may be sometimes produced by a cause on the side of the subject, such as arrhythmia, or a cause due to a measurement apparatus (e.g., noise mixture). Although not illustrated, the biological parameter acquiring section 11 acquires also the ST value for each heart beat, and calculates also the average value of the ST value per minute.

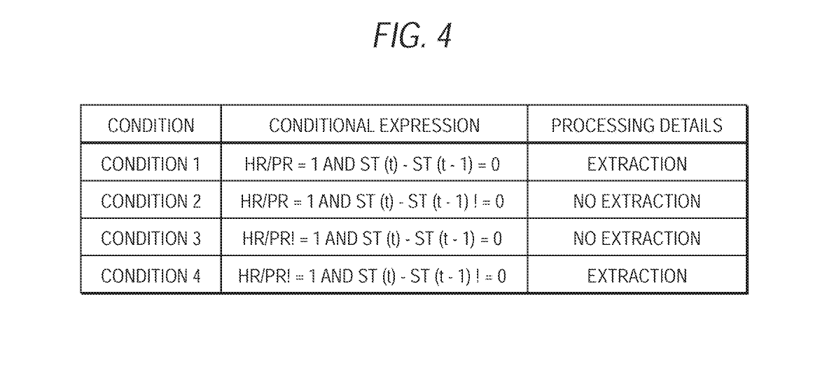

The error excluding section 16 excludes measurement values which cannot be correctly measured by a cause due to the measurement apparatus, and the contact state of a sensor. FIG. 4 shows an example of the exclusion method. The exclusion method will be described with reference to FIG. 4.

In the case where, in measurement values for every minute, no discrepancy exists between the pulse rate (PR) and the heart rate (HR), and also between the ST value and that at one minute before (Condition 1), the error excluding section 16 determines that data are normally measured (necessary measurement values are acquired).

In the case where, in measurement values for every minute, no discrepancy exists between the pulse rate (PR) and the heart rate (HR), and a discrepancy exists between the ST value and that at one minute before (Condition 2), the error excluding section 16 determines that data are not normally measured (necessary measurement values are not acquired). This is caused because a phenomenon that, even though a change of the ST value is due to an electrocardiogram (i.e., the heart), the heart rate (HR) and the pulse rate (PR) do not change is unnatural.

In the case where, in measurement values for every minute, a discrepancy exists between the pulse rate (PR) and the heart rate (HR), and no discrepancy exists between the ST value and that at one minute before (Condition 3), the error excluding section 16 determines that data are not normally measured (necessary measurement values are not acquired). Also this is caused because a phenomenon that, even though a change of the ST value is due to an electrocardiogram (i.e., the heart), the heart rate (HR) and the pulse rate (PR) do not change is unnatural.

In the case where, in measurement values for every minute, a discrepancy exists between the pulse rate (PR) and the heart rate (HR), and also between the ST value and that at one minute before (Condition 4), the error excluding section 16 determines that data are normally measured (necessary measurement values are acquired). It is considered that this is because the ST value is changed by a change in heart ftmction, and a discrepancy between the pulse rate (PR) and the heart rate (HR) is caused by the change (e.g., arrhythmia) in heart function. That is, it seems that this is a state where a certain change due to the subject occurs, and therefore the measurement values are highly possible to be useful in prediction of the heart rate (HR).

The error excluding section 16 extracts only measurement values which satisfy Conditions 1 and 4 in FIG. 4. In the above description, it is assumed that the error excluding section 16 deletes measurement values in accordance with the model of FIG. 4. In an actual process, alternatively, a data processing technique such as the decision tree may be employed. The error excluding section 16 determines whether, in the condition example of FIG. 4, the heart rate (HR) and the pulse rate (PR) are the same (whether HR/PR=1). Alternatively, the determination may be made based on whether the difference of the rates is equal to or smaller than a predetermined value. With respect to the ST value, the error excluding section 16 determines whether there is a discrepancy between the ST value and that at one minute before. Alternatively, the determination may be performed based on whether the difference of the values is equal to or smaller than a predetermined value.

The outlier excluding section 17 excludes outliers from the measurement values which are selected by the error excluding section 16, to perform further data selection. The outlier excluding section 17 may exclude outliers by a technique such as: (1) outliers are excluded after a box plot chart is defined; (2) outliers are excluded as a result of a comparison with a threshold; (3) measurement values of top/bottom X % are excluded; and (4) other techniques. In (2) above, the outlier excluding section 17 determines measurement values which are equal to smaller than, for example, 40, as outliers, and excludes the values. In (3) above, for example, the outlier excluding section 17 determines measurement values of top 5% and those of bottom 5%, as outliers.

Hereinafter, an example of (1) above will be described. The outlier excluding section 17 excludes outliers by using the principle of a box plot chart. For example, the outlier excluding section 17 defines (equal to or smaller than 25 percentile−IQR×1.5) and (equal to or larger than 75 percentile+IQR×1.5) as the thresholds for an outlier, and excludes measurement values. When the outlier excluding section 17 excludes a measurement value, also measurement values related to the value are excluded. In the case where the outlier excluding section 17 excludes the pulse rate (PR) which is measured after ten minutes from the measurement start, as an outlier, for example, also the heart rate (HR) which is measured after ten minutes from the measurement start, and the ST value are excluded.

Preferably, the outlier excluding section 17 repeats the outlier exclusion process using a box plot chart, based on a correlation coefficient r. For example, it is assumed that the outlier exclusion process causes the correlation coefficient r to transit in the following manner

Before outlier exclusion: r=0.680

After first outlier exclusion: r=0.773

After second outlier exclusion: r=0.791

After third outlier exclusion: r=0.802

After fourth outlier exclusion: r=0.799

Preferably, the outlier excluding section 17 repeatedly redefines the box plot chart until the correlation coefficient r has a downward trend. In the above case, the outlier excluding section 17 performs three times the outlier exclusion process (an (n+1) number of processes of producing a box plot chart), and then selects data. The outlier excluding section 17 takes care so that the level of significance cannot be maintained because of the number of data after exclusion.

As described above, the pulse rate (PR) and the heart rate (HR) are values originating from the heart beat, and therefore usually have close values. When outliers are repeatedly excluded based on the correlation coefficient r, therefore, it is possible to extract only measurement values in which the relevance of the rates is high.

The data selecting section 14 supplies the measurement values selected by the error excluding section 16 and the outlier excluding section 17, to the prediction model producing section 15. The prediction model producing section 15 accumulates measurement values of the pulse rate (PR), heart rate (HR), and ST values which are selected by the data selecting section 14, and analyzes the measurement values to produce a regression formula (future prediction model.

As shown in FIG. 3, for example, the prediction model producing section 15 plots the pulse rate (PR) and the heart rate (HR) in a two-dimensional graph, and performs linear regression analysis using the plots, with the pulse rate (PR) being an independent variable, and the heart rate (HR) being a dependent variable. For example, the prediction model producing section 15 produces a regression formula such as the expression (2) below. The expression (3) is a specific example of the expression (2).

[symbol]

= [symbol] + [symbol] ×PR (2)

[symbol]

=25.6+0.701×PR (3)

The prediction model producing section 15 may perform multiple regression analysis in which the pulse rate (PR) and the ST value are used as independent variables, and the heart rate (HR) is used as a dependent variable. A regression formula calculated by multiple regression analysis is indicated by, for example, the expression (4) below. The expression (5) is a specific example of the expression (4).

[symbol]

= [symbol] + [symbol] ×PR+ [symbol] ×ST (4)

[symbol]

=27.0+0.656×PR+72.0×ST (5)

Preferably, the prediction model producing section 15 verifies the correctness of the produced regression formula by using the t-test. Firstly, the prediction model producing section 15 sets a null hypothesis (there is no correlation between the heart rate (FIR) and the pulse rate (PR)). Thereafter, the prediction model producing section 15 sets the significance level, and performs the t-test. If it is determined that the test is significant, the prediction model producing section 15 determines that “It cannot be said that there is no correlation between the heart rate (HR) and the pulse rate (PR).” In this case, the prediction model producing section 15 rejects the null hypothesis, and determines the above regression formula as that the test is significant. The processing details of the t-test may be equivalent to those of a typical statistical processing. Alternatively, the prediction model producing section 15 may use a test technique other than the t-test.

The prediction model producing section 15 may produce the future prediction model of the heart rate (HR) anew, when a fixed time period has elapsed (e.g., every ten minutes) or when the user performs an operation of giving an instruction to producing the model anew (user operation). For example, the prediction model producing section 15 produces a future prediction model which covers a time period from the measurement start to one hour after the start, and which uses measurement values of the pulse rate (PR), heart rate (HR), and ST value for every minute, and thereafter again produces a future prediction model which uses measurement values of the pulse rate (PR), heart rate (HR), and ST value for every minute in a range from one hour after the measurement start to two hours after the start. In this way, the prediction model producing section 15 again produces a future prediction model by using a history of values in a range from the present to a certain time period before. Since a future prediction model is produced anew, the biological information predicting section 12 can perform accurate prediction of the heart rate (HR) with a future prediction model that reflects the current condition of the subject.

The biological information predicting section 12 calculates a future trend (preferably, a future value or risk level) of the heart rate (HR) by using the regression formula (future prediction model) produced by the prediction model producing section 15, and then supplies the calculated future trend to the notifying section 13.

For example, the technique of prediction by the biological information predicting section 12 is as follows. The biological information predicting section 12 refers a history of values of the pulse rate (PR) in a range from the present to a certain time period before (e.g., ten minutes before). Then, the biological information predicting section 12 produces a prediction formula of the pulse rate (PR) from the history of values of the pulse rate (PR). The biological information predicting section 12 performs, for example, regression analysis to produce a prediction formula such as the expression (6) below. In the formula, “t” means t minutes after the current time.

[symbol]

= [symbol] + [symbol] ×t (6)

From the prediction formula (the expression (6)), the biological information predicting section 12 calculates the future value of the pulse rate (PR) at t minutes after the current time (e.g., t=5 or t=10). Then, the biological information predicting section 12 substitutes the calculated pulse rate (future value of the pulse rate (PR) at t minutes after) in the future prediction model (the expression (2) or (3)) to calculate the heart rate (HR) at t minutes after.

In the case where the prediction model producing section 15 performs multiple regression analysis (the expression (4) or (5)), a prediction formula (the expression (7) below) of the ST value is produced from the history of values of the measurement values of the ST value. Then, the biological information predicting section 12 substitutes the future values of the pulse rate (PR) at t minutes after and the ST value in the future prediction model (the expression (4) or (5)) to calculate the heart rate (HR) at t minutes after.

[symbol]

= [symbol] + [symbol] ×t (7)

The prediction model producing section 15 periodically calculates the future value of the heart rate (HR) at t minutes after. The prediction model producing section 15 may calculate the risk level based on the future value. The prediction model producing section 15 informs the notifying section 13 of the calculated risk level and future value of the heart rate (HR).

In the case where the biological parameters show abnormal values, the notifying section 13 outputs an alarm. In the case where the future trend of the heart rate (HR) is not normal as described above (where an abnormal value or an abnormal state is expected in future), the notifying section 13 outputs an alarm even if the current value of the heart rate (HR) is within the normal range.

Here, advantageous effects of the biological information predicting apparatus 1 described above will be described. As described above, the notifying section 13 is configured to provide a notification (preferably, outputs an alarm sound) before the heart rate (HR) shows an abnormal value (even when it is currently within the normal range), in accordance with the prediction by the biological information predicting section 12. A doctor or the like can refer to the notification, and can notice the future risk of a disease as compared with general biological information monitors.

In the present exemplary embodiment, the prediction model producing section 15 produces the future prediction model of the heart rate (HR) based on the history of values of the pulse rate (PR) and the ST value. The prediction model producing section 15 produces the future prediction model by using the history of values of various biological parameters acquired from the subject, and therefore it is possible to produce a future prediction model that is further matches with the subject.

The data selecting section 14 (the error excluding section 16 and the outlier excluding section 17) extracts only measurement values which can ensure the accuracy of a future prediction model, before the prediction model producing section 15 produces the future prediction model. For example, the outlier excluding section 17 produces a box plot chart, and excludes an outlier, thereby extracting only necessary measurement values. Therefore, the prediction model producing section 15 can produce a more accurate future prediction model.

The outlier excluding section 17 repeatedly performs the exclusion of an outlier based on the correlation coefficient r. Therefore, the outlier excluding section 17 can accurately extract only correlated measurement values.

Next, a configuration of a biological information predicting apparatus 1 according to another exemplary embodiment of the present invention will be described. The biological information predicting apparatus 1 of this exemplary embodiment is configured to predict the second biological parameter by using a predifined future prediction model. Features of this exemplary embodiment that are different from those of the exemplary embodiment described above will be described.

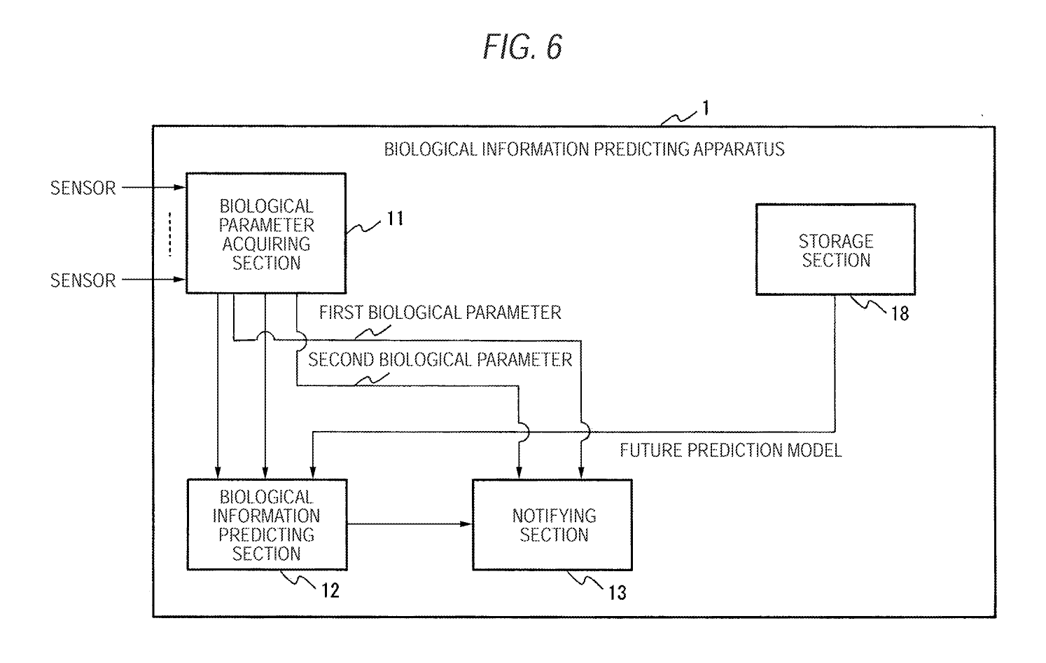

FIG. 6 is a block diagram of the configuration of the biological information predicting apparatus 1 of this exemplary embodiment. The biological information predicting apparatus 1 includes, in addition to the configuration of FIG. 1, a storage section 18 storing a predefined future prediction model.

The storage section 18 is a secondary storage device storing a future prediction model. For example, the storage section 18 may be a device incorporated in the biological information predicting apparatus 1, such as a hard disk drive, or a removable media which can be attached to and detached from the biological information predicting apparatus 1, such as a flash memory.

An example of the future prediction model stored in the storage section 18 will be described. Also in the present example, the first biological parameter is the pulse rate (PR), and the second biological parameter is the heart rate (HR). The future prediction model is similar to, for example, the expression (2). In this exemplary embodiment, the expression is a prediction formula that is predetermined by past rules of thumb. Like the previously described exemplary embodiment, the biological information predicting section 12 refers a history of values of the pulse rate (PR) to calculate the future value of the pulse rate (PR), and substitutes the future value in the expression (2) to calculate the future value of the heart rate (HR).

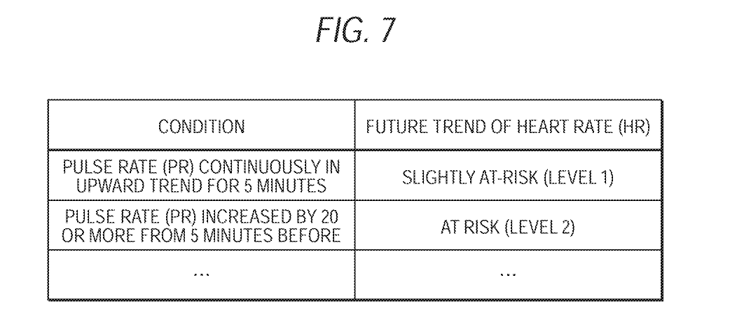

FIG. 7 illustrates a second example of the future prediction model stored in the storage section 18. In the future prediction model, a future risk level of the heart rate (HR) is defined in accordance with the changing trend of the pulse rate (PR). The biological information predicting section 12 compares the history of values of the pulse rate (PR) with the future prediction model (FIG. 7) to detect the risk level of the heart rate (HR). When a possibility that the future trend of the heart rate (HR) becomes abnormal (the risk level is 1 or more) is detected, the biological information predicting section 12 informs the notifying section 13 of this.

The above-described future prediction model is a mere example, and may predict the heart rate (HR) in consideration of a plurality of biological parameters (the ST value and the like). Of course, the model may be defined in a manner other than that described above.

Here, advantageous effects of the biological information predicting apparatus 1 of this exemplary embodiment will be described. As described above, the biological information predicting section 12 predicts the second biological parameter by using a predetermined future prediction model. In other words, the biological information predicting apparatus 1 does not produce a future prediction model during measurement of biological information. Therefore, the biological information predicting apparatus 1 can predict the second biological parameter while reducing the throughput of the apparatus.

While the present invention has been described with reference to certain exemplary embodiments thereof, the scope of the present invention is not limited to the exemplary embodiments described above, and it will be understood by those skilled in the art that various changes and modifications may be made therein without departing from the scope of the present invention as defined by the appended claims.

For example, while the pulse rate (PR) has been described as the first biological parameter and the heart rate (HR) is described as the second biological parameter in the examples described above, the biological parameters are not limited to the pulse rate (PR) and the heart rate (HR). The biological parameters may be other various parameters, such as body temperature, respiration, pulse wave and the like. It is also possible to treat the pulse rate (PR) as the second biological parameter (a biological parameter on which future prediction is conducted).

A part or all of the processes in the biological information predicting section 12, the notifying section 13, and the data selecting section 14 may be implemented as computer programs which operate in the biological information predicting apparatus 1. The programs may be stored in a non-transitory computer readable medium of any one of various types, and then supplied to the computer. The non-transitory computer readable medium includes tangible storage media of various types. Examples of the non-transitory computer readable medium include a magnetic recording medium (e.g., a flexible disk, a magnetic tape, and a hard disk drive), a magneto-optical recording medium (e.g., a magneto-optical disk), a CD-read only memory (CD-ROM), a CD-R, a CD-R/W, a semiconductor memory (e.g., a mask ROM, a programmable ROM (PROM), an erasable PROM (EPROM), a flash ROM, and a random access memory (RAM)). Alternatively, the programs may be supplied to the computer by means of a transitory computer readable medium of any one of various types. Examples of the transitory computer readable medium include an electrical signal, an optical signal, and an electromagnetic wave. The transitory computer readable medium can supply the programs to the computer through a wired communication path such as a metal wire or an optical fiber, or a wireless communication path.

What is claimed is:

1. A biological information predicting apparatus comprising:
a biological parameter acquiring section configured to acquire a first biological parameter and a second biological parameter;
a biological information predicting section configured to predict a future trend of the second biological parameter based on a future prediction model a history of values of the first biological parameter acquired by the biological parameter acquiring section, the future prediction model defining a relationship between a change of the first biological parameter and a change of the second biological parameter; and
a notifying section configured to provide a notification related to the second biological parameter based on a prediction by the biological information predicting section.

2. The biological information predicting apparatus according to claim 1, wherein the notifying section is configured to provide the notification related to the second biological parameter, before the second biological parameter shows an abnormal value, based on the prediction by the biological information predicting section.

3. The biological information predicting apparatus according to claim 1, wherein the biological information predicting section is configured to calculate a risk level as the future trend of the second biological parameter.

4. The biological information predicting apparatus according to claim 1, wherein the notifying section is configured to provide a first notification in a case where the second biological parameter is abnormal and to provide a second notification in a case where the biological information predicting section predicts that the second biological parameter becomes abnormal in future, wherein the first notification and the second notification are different from each other.

5. The biological information predicting apparatus according to claim 4, wherein the first notification includes a first alarm sound, and the second notification includes a second alarm sound, wherein the first alarm sound and the second alarm sound are different from each other.

6. The biological information predicting apparatus according to claim 3, wherein the notifying section is configured to provide different notifications depending on the risk level.

7. The biological information predicting apparatus according to claim 1, further comprising a prediction model producing section configured to perform a regression analysis, based on the history of values of the first biological parameter and a history of values of the second biological parameter that are acquired by the biological parameter acquiring section, to produce a regression formula as the future prediction model.

8. The biological information predicting apparatus according to claim 7, wherein the biological parameter acquiring section is configured to further acquire a third biological parameter, and
wherein the prediction model producing section is configured to produce the regression formula, with the history of values of the first biological parameter and a history of values of the third biological parameter being independent variables, and the second biological parameter being a dependent variable.

9. The biological information predicting apparatus according to claim 7, wherein the prediction model producing section is configured to produce, each time a constant time period elapses or when a user operation is performed, the regression formula anew using the history of values of the first biological parameter and the history of values of the second biological parameter.

10. A biological information predicting method comprising:
acquiring a first biological parameter and a second biological parameter;
predicting a future trend of the second biological parameter based on a future prediction model and a history of acquired values of the first biological parameter, the future prediction model defining a relationship between a change of the first biological parameter and a change of the second biological parameter; and
providing a notification related to the second biological parameter based on the predicted future trend.

11. A non-transitory computer readable medium storing a program that, when executed by a computer, causes the computer to execute a method comprising:
acquiring a first biological parameter and a second biological parameter;
predicting a future trend of the second biological parameter based on a future prediction model and a history of acquired values of the first biological parameter, the future prediction model defining a relationship between a change of the first biological parameter and a change of the second biological parameter; and
providing a notification related to the second biological parameter based on the predicted future trend.
